(19) United States
(12) Patent Application Publication
(10) Pub. No.: US 2004/0260201 A1
(43) Pub. Date: Dec. 23, 2004

(54) Cytology brush with releasable end portion

(76) Inventor: Richard L. Mueller JR., Jackson, WY (US)

Correspondence Address: OLSON & HIERL, LTD., 36th Floor, 20 North Wacker Drive, Chicago, IL, 60606, US

(21) Appl. No.: 10/602,171

(22) Filed: Jun. 23, 2003

(51) Int. Cl. A61B 10/00

(52) U.S. Cl. 600/569

(57) ABSTRACT

A cytology brush suitable for conducting a brushing biopsy within a mammary duct of a human patient is provided. The cytology brush comprises a handle and a detachable or releasable brush. After a brushing biopsy the brush end can be released and retained in the collection vessel. Optionally, a dispensable biopsy site marker is carried by the brush and can be left in place after biopsy.

### TECHNICAL FIELD OF THE INVENTION

The invention relates to a device for obtaining cytology samples. More specifically, the invention relates to a biopsy brush with a detachable or releasable brush component.

### BACKGROUND OF THE INVENTION

Breast cancer is one of the health threats most feared by women, and is the most common form of cancer in women, and in rare instances, men. A key to treatment is early detection. For example, an annual mammogram is a method that has been used in hopes of early detection of breast cancer. One problem with mammography is that such an imaging technique can only find breast cancer once it has taken form. All too often, breast cancer is discovered at a stage that is too far advanced, when therapeutic options and survival rates are severely limited. As such, more sensitive and reliable methods and devices are needed to detect cancerous, pre-cancerous, and other cancer markers of the breast at an early stage. Such methods and devices could significantly improve breast cancer survival.

Some methods of detecting breast cancer are based on the fact that a majority of instances of breast cancer begins in the lining of mammary ducts. Studies have shown that fluid within the mammary duct contains high levels of breast cancer markers, and that an estimated 80%-90% of all breast cancers occur within the intraductal epithelium of the mammary glands. Fluid within the breast ducts contains an assemblage and concentration of hormones, growth factors and other potential markers comparable to those secreted by, or acting upon, the surrounding cells of the alveolar-ductal system. Likewise, mammary fluid typically contains cells and cellular debris, or products that can also be used in cytological or immunological assays. Procedures for obtaining such samples include ductal lavage, expression or aspiration of mammary duct fluid, and collection of mammary duct discharge. Ductal lavage comprises the introduction of a rinsing solution into a mammary duct, such as a saline solution or the like, and the collection of the solution along with any cells and cellular debris from the mammary duct.

It is sometimes desirable to increase the yield of cells and cellular debris from a ductal lavage through use of an intraductal brush and a brushing biopsy procedure to loosen and dislodge cellular material from the intraductal epithelium of the mammary glands. In addition to the material that is dislodged and collected through ductal lavage or other methods of duct fluid collection, such material may also be found entrapped on the biopsy brush itself. As such, it is desirable to analyze the material retained on the bristles of a biopsy. It is advantageous to be able to submit a vial of fluid collected from a ductal lavage along with the brush tip or head together to avoid any confusion regarding the samples. Typically, however, biopsy brushes have a rigid shaft attached to a brush head that is often made of stainless steel and is difficult to sever with instruments commonly found in a doctor's office because of their strength and resistance to shearing.

### SUMMARY OF THE INVENTION

A cytology brush suitable for conducting a brushing biopsy and having a removable or detachable end portion is provided. The cytology brush is sized for entry into the mammary duct of a human patient and comprises a releasable bristle shank having bristles thereon. The bristles are of sufficient stiffness to loosen cells lining the mammary duct, such as within the intraductal epithelium, without substantial traumatization of mammary duct tissue. The bristles are preferably made of nylon. Preferably, the bristle shank has an atraumatic tip, such as a tapered or rounded tip, or a tip of a relatively soft material.

A brush handle is operatively connected to the bristle shank to enable a practitioner to manipulate the brush and control the brushing action by the bristles. The brush handle and bristle shank are preferably made of a rigid material, such as stainless steel or a sterilizable plastic material. The handle and bristle shank are operatively connected to one another, but are also detachable from one another, such as via a coupling. The coupling may take various forms, e.g., a threaded connection between the shank and the handle, a friction connection, such as a socket or a collar, between the shank and the handle, or the like. The coupling may alternatively be a claw assembly carried by the brush handle that releasably engages the bristle shank. As yet another alternative, the coupling can be a helix or corkscrew at the distal end of the handle that releasably engages the bristle shank.

The cytology brush may also include a dispensable biopsy site marker for marking the location of a brushing biopsy. For example, an expanding marker coil can be introduced into the mammary duct by the cytology brush. In so doing, if further analysis of the region or additional biopsy samples are desired, the location of the previous brushing biopsy can be readily revisited.

### BRIEF DESCRIPTION OF THE DRAWINGS

In the drawings,

FIG. 1 is a side view of a cytology brush embodying the present invention with a coupling shown in section;

FIG. 2 is side view of the cytology brush of FIG. 1 in a mammary duct with an introducer shown partially in section;

FIG. 3 is a partial cross sectional view of an alternate embodiment of a cytology brush with a threaded coupling;

FIG. 4 is a partial side view of an alternate embodiment of a cytology brush with a corkscrew coupling;

FIG. 5 is a partial cross sectional view of an alternate embodiment of a cytology brush with a tapered brush handle and recessed bristle shank;

FIGS. 6A and 6B are cross sectional views of yet another embodiment of a cytology brush with a claw engageable with the bristle shank;

FIGS. 7A and 7B are cross sectional views of an embodiment of a cytology brush with a claw engageable with the bristle shank, an expandable coil biopsy site marker, and a detent;

FIG. 8 is a cross section of the detent of FIG. 7B taken along line 8-8;

FIG. 9 is a cross section of an alternate embodiment of a detent; and

FIG. 10 is a cross section of another alternate embodiment of a detent.

### DESCRIPTION OF PREFERRED EMBODIMENTS OF THE INVENTION

The invention disclosed herein is susceptible of being embodied in many different forms. Shown in the drawings and described herein below in detail are preferred embodiments of the invention. It is to be understood, however, that the present disclosure is an exemplification of the principles of the invention and does not limit the invention to the illustrated embodiments.

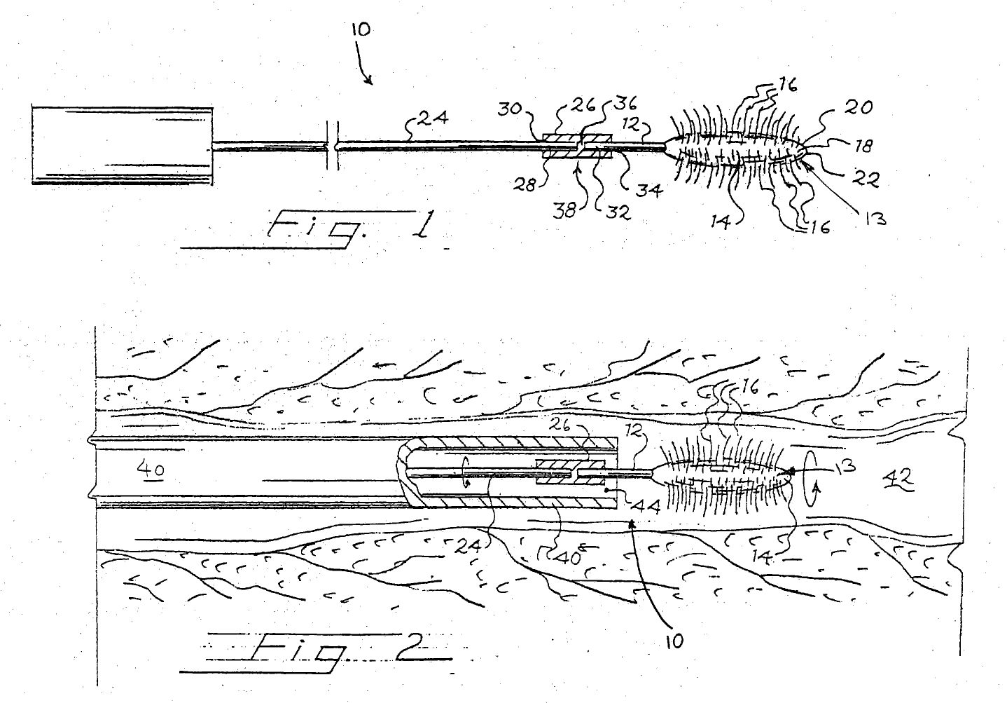

An embodiment of a cytology brush 10 is shown in FIG. 1. Brush 10 comprises a releasable bristle shank 12 having a bristle structure 13 which is constituted by bristle backbone 14 and bristles 16 thereon. In order to lessen the possibility of trauma to a mammary duct, the bristle structure 14 includes an atraumatic tip 18 that preferably includes a tapered portion 20 and a rounded tip 22. Bristles 16 are also made of a flexible, relatively soft material, such as nylon, which enables brushing of the intraductal epithelium, but does not damage the mammary duct wall itself. Bristles 16 may also have curved ends to further lessen any traumatic effect of the brushing biopsy procedure.

A brush handle 24 is operatively but releasably connected to the bristle shank 12 so as to enable a practitioner to control the brushing action of the bristles. A friction coupling such as collar 26 is provided to removably attach the bristle shank 12 to the brush handle 24. To that end distal end portion 28 of brush handle 24 is received in sleeve 30, and proximal end portion 32 of bristle shank 12 is received in sleeve 34. Preferably, collar 26 is formed of a plastic material that can be severed by a knife or scissors, if desired. A partition or septum 36 may separate sleeve 30 from sleeve 34, if desired, and can provide a convenient region of severance when shank 12 is to be separated from handle 24. Preferably, a marking 38 is provided on the outer circumference of collar 26 to provide a visual indicator of the location of partition or septum 36. Alternatively, partition or septum 36 can be a weakened region between sleeves 30 and 34 so that shank 12 and sleeve 34 can be severed by a twisting action.

FIG. 2 shows brush 10 positioned in a mammary duct. In order to position brush 10 at the desired location, an introducer 40 is first inserted into a mammary duct 42. An endoscope (not shown) may be passed through the introducer 40 to enable the practitioner to position the end of the introducer 40 at the desired region in the mammary duct. The endoscope is withdrawn, and thereafter brush 10 is inserted through introducer 40. Brush 10 is then extended beyond the open distal end 44 of the introducer 40 such that bristle structure 13 comprising backbone 14 and bristles 16 can be brought into contact with the intraductal epithelium. Brush 10 may be either rotated or moved axially such that bristles 16 sweep, scrape or brush against the intraductal epithelium or walls of the mammary duct 42, thereby dislodging material for an assay. Brush 10 may then be withdrawn from the mammary duct 42, and a rinsing solution introduced into the mammary duct via the introducer 40 for a ductal lavage procedure. The rinsing solution and any material contained or suspended therein can then be collected via the introducer 40 for further analysis.

It is likely that some material of cytological interest is also present on the bristles 16. As such, it is desirable to also examine the bristle structure 13 for entrapped material. To that end, brush handle 24 can be disengaged from bristle shank 12 by disengaging either the distal end portion 28 of brush handle 24, the proximal end portion 32 of bristle shank 12 from collar 26, or by severing collar 26. The bristle structure 13 is then retained for analysis. The bristle shank 12 can be grasped and the brush handle 24 pulled from the collar 26. Grasping of the bristle shank 12 can be effected with any suitable clamping device, tweezers, pliers or the like (not shown), so as to avoid contamination of any cytology specimen on the bristles through contact. Alternatively, a practitioner may sever brush handle 24 from bristle shank 12 in any convenient manner, e.g., by cutting partition 36, in the region of marking 38 (FIG. 1). Since partition 36 is preferably made of a plastic material, it may readily be cut using clips or scissors typically found in doctor's offices. The bristle shank 12 and bristle structure 14 together with bristles 16 can then be analyzed for the presence of markers having diagnostic significance.

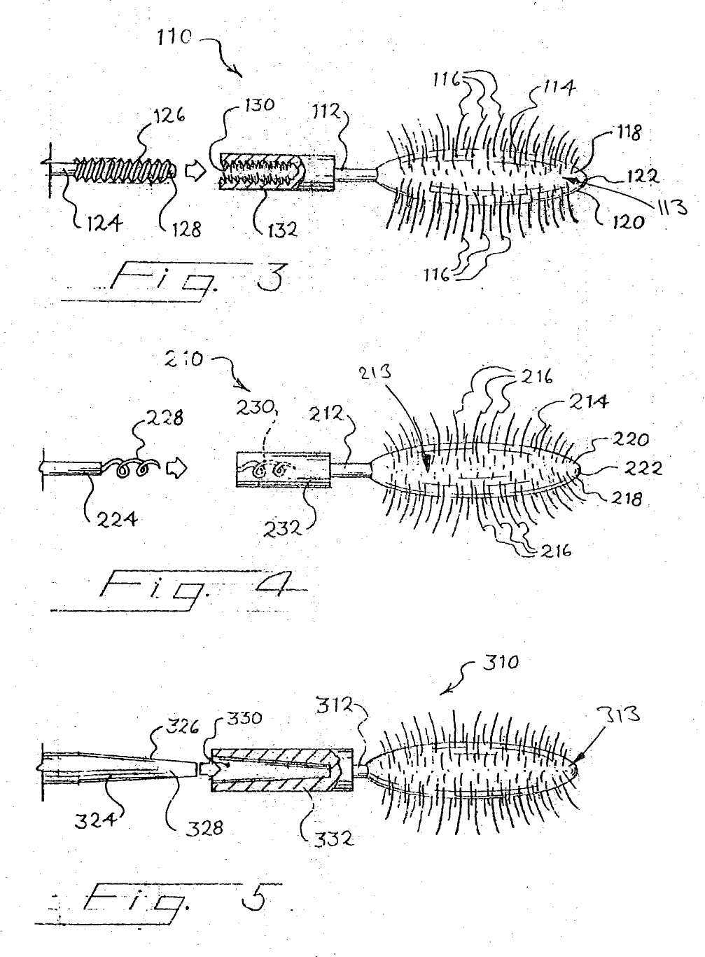

An alternate embodiment of a cytology brush 110 is shown in FIG. 3. Similar to the previous embodiment, but having a different coupling between the shank and the handle, the brush 110 comprises a bristle shank 112 having a bristle structure 113 and backbone 114 with an atraumatic tip 118 that has a tapered portion 120 and a rounded tip 122. The bristle backbone 114 carries bristles 116. In this particular embodiment, brush handle 124 is directly connected to bristle shank 112 via a coupling with external threads 126 on distal end portion 128 and internal threads 130 on proximal end portion 132. The proximal end portion 132 of bristle shank 112 is removably attached to the distal end portion 128 of the brush handle 124 when threadably engaged therewith. In order to uncouple brush handle 124 from bristle shank 112 after a brushing biopsy is completed, brush handle 124 is rotated relative to bristle shank 112. During rotation, the bristle shank can be held in place with any suitable clamping device, tweezers, pliers, or the like. If desired, the threaded coupling shown in FIG. 3 can be replaced with a luer-lock type coupling.

Yet another alternate embodiment of a cytology brush 210 is shown in FIG. 4. In this embodiment brush 210 again comprises a bristle shank 212 having a bristle structure 213 with backbone 214 and bristles 216 thereon, and an atraumatic tip 218. Brush handle 224 is connected to bristle shank 212 via a coupling with a distal end portion 228 having a helical or corkscrew configuration. Distal end portion 228 is received in complementary helical passage 230 defined by proximal end portion 232 of bristle shank 212. Uncoupling brush handle 224 from bristle shank 212 after a brushing biopsy is completed may be accomplished in a manner similar to that discussed above with respect to the embodiment shown in FIG. 3.

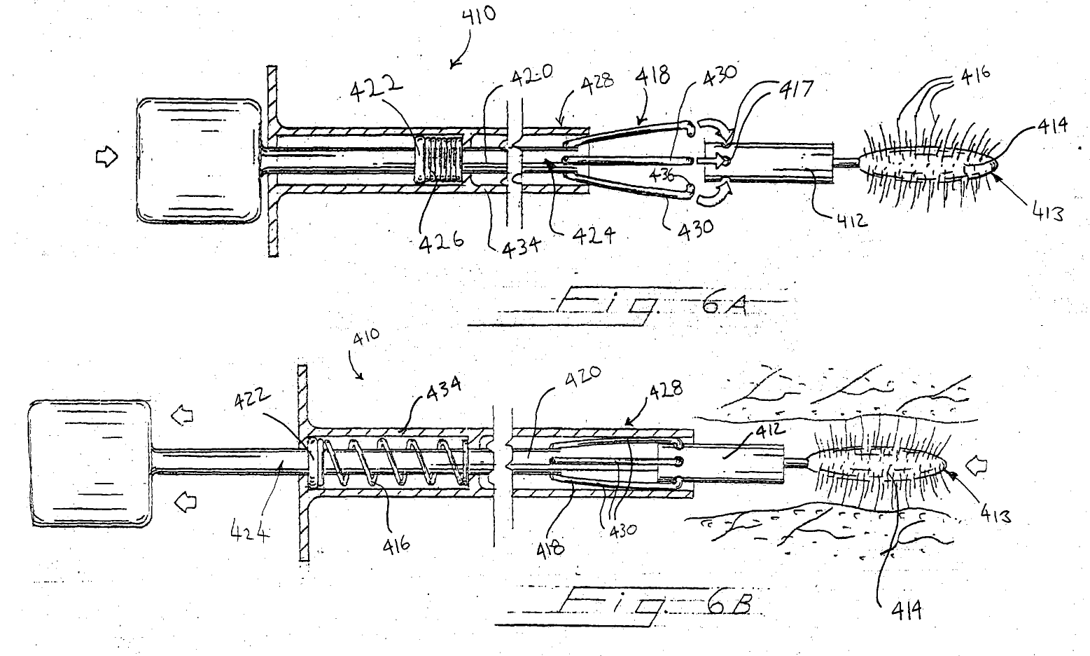

A further example of a friction coupling for removably securing a brush handle to a bristle shank is shown in FIG. 5. Brush 310 includes bristle structure 313 and a brush handle 324 having a distal end portion 328 defining a taper 326. The proximal end portion 332 of bristle shank 312 defines a complementary tapered recess or socket 330 that frictionally engages distal end portion 328 therein. A luer taper is preferred for this purpose. Disengagement of the brush handle 324 from the bristle shank 312 is accomplished by grasping the bristle shank 312 and withdrawing brush handle 324 from bristle shank 312 with a twisting motion. Although not shown, a tapered distal end portion of a brush handle may also include a threaded portion that is suitable for engaging threads formed within the recess in the bristle shank.

A still further embodiment of a coupling between a brush handle and a bristle shank is shown in FIGS. 6A and 6B. Brush 410 comprises bristle shank 412 attached to brush handle 424 and a bristle structure 413 including backbone 414 and bristles 416 thereon. Bristle shank 412 further defines peripheral locking recesses 417 about the bristle shank 412. Brush handle 424 is slidably received within an elongated housing such as tubular housing 434 and is equipped with a claw assembly 418 operatively connected by a shaft 420 that carries a plunger 422 that abuts coil spring 426. Claw assembly 418 comprises a plurality of prongs 430 that are engageable with locking recesses 417 on bristle shank 412. Coil spring 426 surrounds shaft 424 and biases claw assembly 418 so as to engage bristle shank 412 and retract claw assembly 418 into housing 434. Prongs 430 of claw assembly 418 are radially outwardly biased, however. As claw assembly 418 is drawn within housing 434, ends 436 of prongs 430 contact the inner wall of the housing and are urged toward one another to grasp and hold the proximal end of bristle shank 412. Prior to insertion of brush 410 into the mammary duct, claw assembly 418 secures bristle shank 412 to brush handle 424 so that the bristles can be readily manipulated within the mammary duct. After a brushing biopsy is performed and the brush 410 is withdrawn from the mammary duct, bristle shank 412 is disengaged from brush handle 424 by depressing plunger 422 to cause claw assembly 418 to exit the distal end portion 428 of housing 434 and release bristle shank 412. Brush handle 424 is preferably made of sterilizable materials such that it may be used repeatedly by replacing the bristle carrying portion.

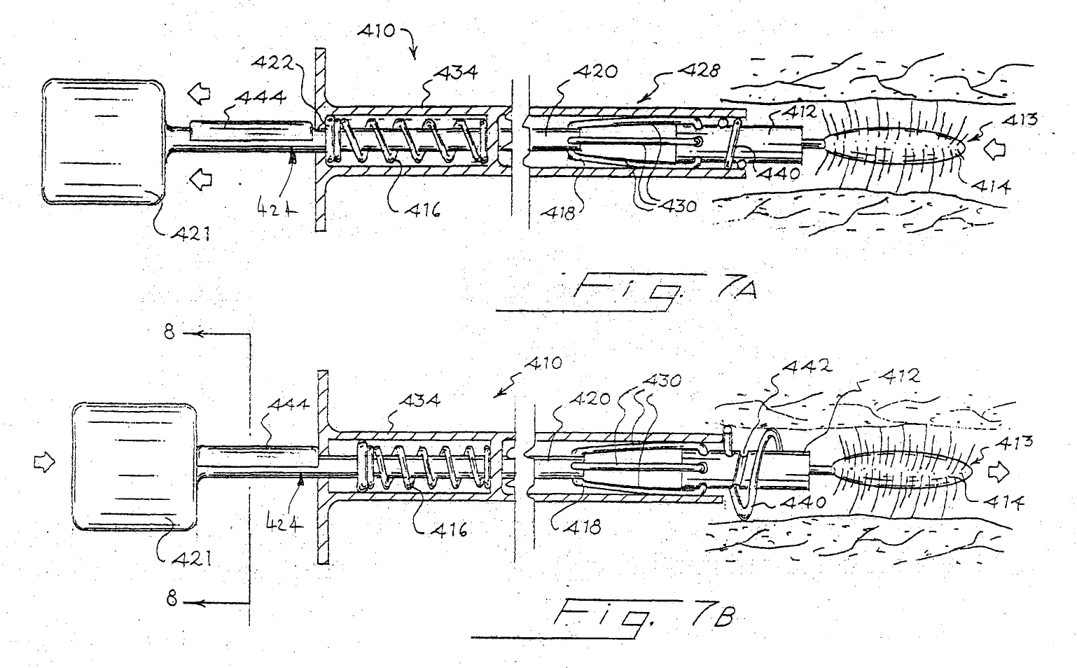

Another optional feature of the present invention is an opaque biopsy site marker carried by and dispensable from the cytology brush. Such a marker may be left behind in the mammary duct after a biopsy to mark the location of the brushing biopsy in the event that later analysis or inspection of the location or additional procedures, such as another brushing biopsy, are desired. An example of a cytology brush with a biopsy site marker is shown in FIGS. 7A and 7B. The brush 410 is the same brush as that shown in FIGS. 6A and 6B, but with some additional features. Circumscribing the bristle shank 412 is a radio opaque marker such as circumferentially outwardly biased coil 440. Preferably, coil 440 is made of titanium, stainless steel, or other like biologically inert material that can be detected subsequently by X-ray, ultrasound, and the like imaging techniques. As shown in FIG. 7A, coil 440 is positioned distally of prongs 430 and is seated in groove 442 (FIG. 7B). As brush 410 is extended, coil 440 is urged past distal end portion 428 is dispensed into the mammary duct. Coil 440 expands circumferentially outwardly against the mammary duct wall after exiting distal end portion 428, and is frictionally secured therewith; as shown in FIG. 7B, thereby marking the location of the brushing biopsy. The bristle structure 413 can be passed through coil marker 440 after the brushing operation as the bristle structure 413 is withdrawn into the distal end portion 428.

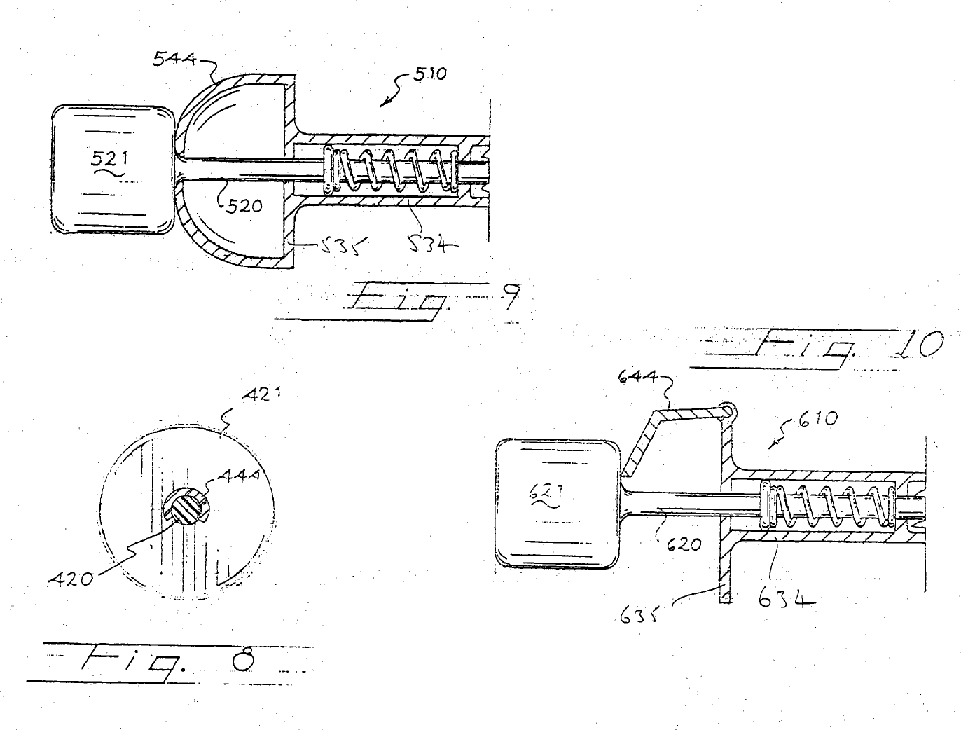

In order to prevent inadvertent release of the bristle structure 413 from brush 410 as the biopsy site marker is positioned a detent, such as a partial flexible sleeve can be provided about a proximal end portion of shaft 420 which serves as part of brush handle 424. Referring to FIGS. 7A, 7B, and 8, flexible sleeve 444 is preferably made of a flexible material, such as plastic, is clipped onto, and partially circumscribes shaft 420 of brush handle 424 which terminates at the proximal end in button 421. Greater than one-half of the circumference of shaft 420 is circumscribed by sleeve 444 such that sleeve 444 is removably clipped onto shaft 420 and limits the axial travel of brush handle 424 until removed. Sleeve 444 is slidable along the shaft 420 such that as button 421 is urged towards housing 434 sleeve 444 moves along the shaft but limits the extent to which button 421 and shaft 420 can be displaced axially. After the brush 410 is withdrawn from the mammary duct, the sleeve 444 can be removed to allow the shaft 420 to be extended such that claw 418 releases bristle shank 412.

Other variants of a detent are shown in FIGS. 9 and 10. In FIG. 9, an integrally formed detent having the structure of a hollow dome 544 is shown at the proximal end of brush 510. Dome 544 is made of a flexible material, such as an elastomer, and shaft 520 is slidable relative thereto. Dome 544 is integral with flange 555 of housing 534. As button 521 is urged to contact dome 544, as shown in FIG. 9, the practitioner is provided with tactile feedback inasmuch as the dome, when encountered by button 521, provides resistance to the button travel. The encountered resistance provided by dome 544 can be overcome by application of additional force in the axial direction after the shaft 520 is withdrawn from the mammary duct, and the bristle structure is to be released. Dome 544 can be vented, if necessary.

In FIG. 10, the detent on cytology brush assembly 610 is provided by arm 644 which is pivotably mounted in hinged relationship with radially outwardly extending flange 635 on housing 634. Similar to the embodiment shown in FIGS. 7A and 7B, shaft 620 is slidably received in housing 634 and arm 644 prevents inadvertent over-extension of the brush by abutting button 621 at the proximal end of shaft 620. After the brushing biopsy is completed, the arm 644 is pivoted away from engagement with button 621 such that the bristle structure (not shown) can be released from the brush handle.

Thus far, each embodiment of the detent has been discussed with respect to the claw coupling. However, a detent may be included in any of the embodiments previously discussed. For example a housing can be included to protect the coupling during use. A detent, such as described hereinabove may be used to prevent the coupling from being extended past the distal end of the housing until it is desired to uncouple the bristle structure from the handle.

The foregoing description is to be taken as illustrative, but not limiting. Still other variants within the spirit and scope of the present invention will readily present themselves to those skilled in the art.

I claim:

1. A cytology brush, sized for entry into a mammary duct of a human patient and comprising:
a bristle shank;
a bristle structure on the shank and having bristles of sufficient stiffness to loosen cytology material lining the mammary duct without substantial traumatization of mammary duct tissue;
a brush handle; and
a coupling for releasably attaching the bristle shank to the brush handle.

2. The cytology brush in accordance with claim 1, wherein the bristle shank has an atraumatic tip.

3. The cytology brush in accordance with claim 1, wherein the bristles are nylon bristles.

4. The cytology brush in accordance with claim 1, wherein the coupling is a threaded connection between the shank and the handle.

5. The cytology brush in accordance with claim 1, wherein the coupling is a friction connection between the shank and the handle.

6. The cytology brush in accordance with claim 5, wherein the friction connection is a collar.

7. The cytology brush in accordance with claim 1, wherein the coupling is a claw carried by the brush handle and releasably engaging the bristle shank.

8. The cytology brush in accordance with claim 1, wherein the coupling is a corkscrew in the handle and releasably engages the bristle shank.

9. The cytology brush in accordance with claim 1, further including an elongated housing for the brush handle and a biopsy site marker releasably carried by the cytology brush in the elongated housing.

10. The cytology brush of claim 9, wherein the biopsy site marker is an outwardly biased coil releasably mounted to the bristle shank.

11. The cytology brush of claim 1, further including a detent for limiting axial travel of the brush handle within the elongated housing.

12. The cytology brush of claim 11, wherein the detent is removably secured to the brush handle.

13. The cytology brush of claim 11, wherein the brush handle is slidably mounted in the elongated housing and the detent is integral with the elongated housing.

14. The cytology brush of claim 11, wherein the brush handle is slidably mounted in the elongated housing and the detent is mounted to the elongated housing in a hinged relationship therewith.

15. A cytology brush, sized for entry into a mammary duct of a human patient and comprising:
a bristle shank;
a bristle structure on the shank and having bristles of sufficient stiffness to loosen cells lining the mammary duct without substantial traumatization of mammary duct tissue; and
a detachable brush handle operably associated with the bristle shank.

16. The cytology brush of claim 15, wherein the brush handle is connected to the bristle shank by a detachable coupling.

17. The cytology brush in accordance with claim 16, wherein the coupling is a threaded connection between the shank and the handle.

18. The cytology brush in accordance with claim 16, wherein the coupling is a friction connection between the shank and the handle.

19. The cytology brush in accordance with claim 16, wherein the friction connection is a collar.

20. The cytology brush in accordance with claim 16, wherein the coupling is a claw carried by the brush handle and releasably engaging the bristle shank.

21. A cytology brush, sized for entry into a mammary duct of a human patient and comprising:
a bristle shank;
a bristle structure on the shank with bristles of stiffness sufficient to loosen cytological material lining the mammary duct without substantial traumatization of mammary duct tissue;
a detachable brush handle for the bristle shank; and
a dispensable, opaque marker carried by the cytology brush.

22. The cytology brush in accordance with claim 21 and further provided with a detent operably associated with the bristle shank for preventing inadvertent release of the detachable brush handle.

23. The cytology brush in accordance with claim 21 wherein the detent is mounted to the detachable brush handle.

24. The cytology brush in accordance with claim 21 wherein the brush handle is slidably received within a housing and the detent is situated on the housing for operable engagement with the brush handle.

25. The cytology brush in accordance with claim 24 wherein the detent is a pivotable arm on the housing.

26. The cytology brush in accordance with claim 24 wherein the marker is a titanium coil carried on the bristle shank.

27. The cytology brush in accordance with claim 24 wherein the housing is a tubular housing.
